(19) United States
(12) Patent Application Publication
(10) Pub. No.: US 2010/0016599 A1
(43) Pub. Date: Jan. 21, 2010

(54) Process for Preparing Fexofenadine

(76) Inventors: Giuseppe Motta, Milano (IT); Domenico Vergani, Biassono (IT)

Correspondence Address: PROPAT, L.L.C., 425-C SOUTH SHARON AMITY ROAD, CHARLOTTE, NC, 28211-2841, US

(21) Appl. No.: 12/374,688

(22) PCT Filed: Jul. 25, 2007
(86) PCT No.: PCT/IT2007/000526

§ 371 Date: Mar. 24, 2009

(30) Foreign Application Priority Data
Jul. 27, 2006 (IT) MI2006A001491

(51) Int. Cl. C07D 211/34; C07C 69/76

(52) U.S. Cl. 546/239; 560/105

(57) ABSTRACT

A process for preparing fexofenadine is described that includes the purification of 4-[4-chloro-1-oxobutyl]-2,2-dimethylphenyl acetic acid alkyl ester by means of suspension in a hydrocarbon, preferably n-heptane. The compound thus obtained is dissolved in a suitable solvent and condensed with azacyclanol to give the compound shown below

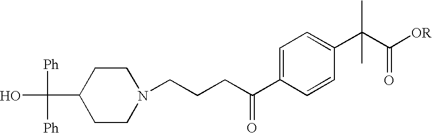

where R is an alkyl radical, which is then hydrolysed and reduced to give fexofenadine.

The object of the present invention is a process for preparing fexofenadine comprising the purification of 4-[4-chloro-1-oxobutyl]-2,2-dimethylphenyl acetic acid methyl ester; more in detail, the present invention concerns a process for preparing fexofenadine, the formula of which is shown below

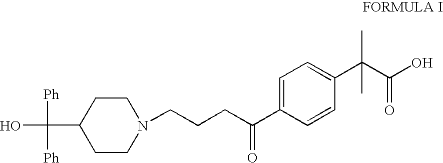

from 4-[4-chloro-1-oxobutyl]-2,2-dimethylphenyl acetic acid alkyl ester the formula of which is also that shown below

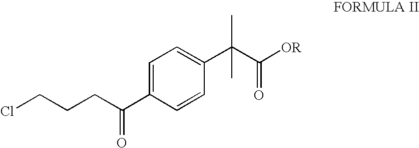

where R is alkyl, preferably C₁-C₄, still more preferably methyl.

The process of the present invention comprising the purification of the compound of formula II by means of suspension of this compound in a hydrocarbon.

In this process, the compound of FORMULA II is suspended at low temperature in a hydrocarbon and filtered after solidification. The compound thus obtained is dissolved in a suitable solvent and condensed with azacyclanol, the formula of which is shown below

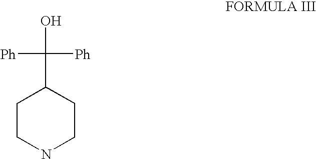

to give the compound shown below

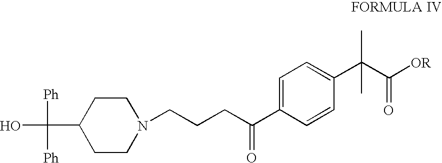

which is then hydrolysed and reduced to give fexofenadine.

### PRIOR ART

The presence in the intermediate of FORMULA II of the isomer of FORMULA V, shown below, has always been one of the most critical factors in the synthesis of fexofenadine.

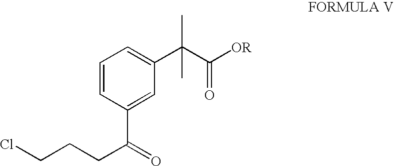

In patent literature, various methods of separating the isomers of FORMULA II and V have been published which provide for their transformation into the products of FORMULA VI and VII, as described for example in U.S. Pat. No. 6,548,675.

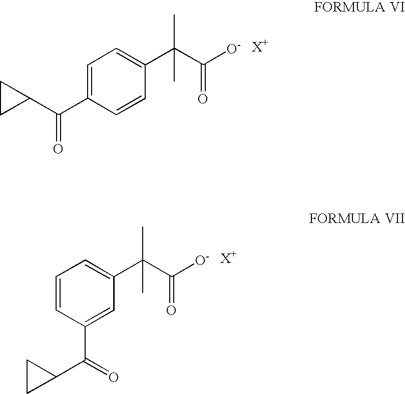

The separation of the two products by crystallisation and the retransformation of the product of FORMULA VI into that of FORMULA II, thus obtained, in a substantially pure form.

### DESCRIPTION OF THE INVENTION

During the development of a synthesis method for fexofenadine, we have surprisingly discovered that the compound of FORMULA II may be purified from the compound of FORMULA V and from other impurities by suspension of the mixture to be purified in an apolar organic solvent. Such solvent is preferably an alkyl-type hydrocarbon, such as for example a compound or mixture of compounds of formula CₙH₂ₙ₊₂, straight and/or branched, where n varies between 5 and 12; the preferred hydrocarbon is n-heptane.

The mixture of the two isomers II and V, which at room temperature is a dense oil, is added dropwise into a reactor containing the above-mentioned hydrocarbon solvent and the mixture is left under stirring at low temperature.

More in detail, such hydrocarbon solvent is normally used in quantities of 2-50 volumes in relation to the mixture to be purified. The mixture thus obtained is then left under stirring for a period of 1-12 hours at a temperature in the range −80-10° C.

The compound of FORMULA II is obtained as a solid while the impurities, and in particular isomer V, remain dissolved in the solvent. The suspension is cold-filtered and the product of FORMULA II can be recovered as a solid and stored as such (at a preferred temperature of about 4° C.) or dissolved in a solvent and directly used in the condensation reaction with azacyclanol.

This reaction is known in the art and described for example in U.S. Pat. No. 4,254,129, incorporated here for reference; preferably, it is normally carried out in an aprotic organic solvent, preferably of a ketone-type, still more preferably methylisobutylketone (MIBK); the temperature is preferably between 40° C. and the reflux temperature of the reaction mixture and the reaction is carried out over a period of about 8-24 hours.

The condensation product is then hydrolysed and reduced to fexofenadine.

The examples which follow are purely illustrative and non limiting of the invention.

### Example 1

100 g of 4-[4-chloro-1-oxobutyl]-2,2-dimethylphenyl acetic acid methyl ester with an HPCL purity of 90% and a 6.5% content of meta isomer are added dropwise in a flask containing 2 litres of heptane at −20° C. under stirring. A suspension is obtained, which is filtered at −20° C. 65 g of purified product with an HPLC purity of 98.9% and a 0.6% content of meta isomer, which is stored as a solid at 4° C., are obtained.

### Example 2

100 g of 4-[4-chloro-1-oxobutyl]-2,2-dimethylphenyl acetic acid methyl ester with an HPCL purity of 90% and a 6.5% content of meta isomer are added dropwise in a flask containing 2 litres of hexane at −30° C. under stirring. A suspension is obtained, which is filtered at −30° C. 66 g of purified product with an HPLC purity of 98.6% and a 0.8% content of meta isomer, which is stored as a solid at 4° C., are obtained.

### Example 3

100 g of 4-[4-chloro-1-oxobutyl]-2,2-dimethylphenyl acetic acid methyl ester with an HPCL purity of 90% and a 6.5% content of meta isomer are added dropwise in a flask containing 2 litres of isooctane at −50° C. under stirring. A suspension is obtained, which is filtered at −50° C. 68 g of purified product with an HPLC purity of 98.9% and a 0.5% content of meta isomer, which is stored as a solid at 4° C., are obtained.

### Example 4

In a 1-litre, 4-necked flask, 50 g of purified 4-[4-chloro-1-oxobutyl]-2,2-dimethylphenyl acetic acid methyl ester obtained in example 1, 38 g of azacyclanol, 18 g of sodium bicarbonate, 250 ml of MIBK and 50 ml of water are loaded. The mixture is heated at reflux and kept under stirring for about 24 hours. Once the reaction is terminated, the mixture is cooled down, 200 ml of water are added and the phases are separated.

The organic phase is concentrated under vacuum to 50 ml. A white precipitate is obtained, which is filtered and dried under vacuum. 63 g of 4-[4-[4-(hydroxydiphenylmethyl)-1-piperidyl]-1-oxobutyl]-α,α-dimethylbenzeneacetic acid-methyl ester are obtained.

### Example 5

In a four-necked flask equipped with a mechanical stirrer, 100 g of 4-[4-[4-(hydroxydiphenylmethyl)-1-piperidyl]-1-oxobutyl]-α,α-dimethylbenzeneacetic acid-methyl ester obtained according to example 2, 600 ml of methanol and 60 ml of 30% sodium hydroxide are loaded. The mixture is heated at reflux and kept under stirring for about 5 hours. When the ester is completely hydrolysed, 10 g of 5% palladium on carbon are loaded into the reactor and are hydrogenated at 50° C. and 6 bar pressure until the complete conversion of the benzylketone into alcohol. Once the reaction is completed, the catalyst is filtered and the fexofenadine is precipitated by adjusting the pH to 5-8 with acetic acid. The solid obtained is filtered and dried under vacuum at 65° C.

85 g of crude fexofenadine are obtained on average with HPLC purity >99%; meta isomer <0.2%.

### Example 6

In a four-necked flask equipped with a mechanical stirrer, 100 g of 4-[4-[4-(hydroxydiphenylmethyl)-1-piperidyl]-1-oxobutyl]-α,α-dimethylbenzeneacetic acid-methyl ester obtained according to example 2, 600 ml of methanol and 130 ml of 30% sodium hydroxide are loaded. The mixture is heated at reflux and kept under stirring for about 2 hours. When the ester is completely hydrolysed, the solution is cooled down and 7 g of sodium borohydride are added. The reaction solution is heated again at 50° C. and kept at this temperature until the complete conversion of benzylketone into alcohol. Once the reaction is completed, 10 ml of acetone are added, it is left under stirring for 30 minutes, it is cooled down and the fexofenadine is precipitated by adjusting the pH to 5-8 with acetic acid. The solid obtained is filtered and dried under vacuum at 65° C.

85 g of crude fexofenadine are obtained on average with HPLC purity: 90%; meta isomer <0.2%.

1. A process for separating a compound of formula 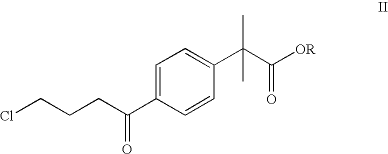
from the corresponding isomer of formula 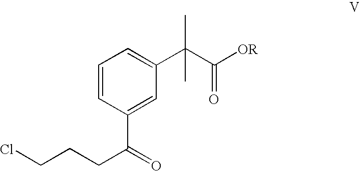
where R is alkyl,
said process comprising adding a mixture of the two isomers II and V to an alkyl hydrocarbon with the resulting precipitation of the isomer of formula II,
wherein said alkyl hydrocarbon is a compound or a mixture of compounds of formula CₙH₂ₙ₊₂, straight and/or branched, where n varies between 5 and 12.

2. A process according to claim 1, wherein R is a C₁-C₄ alkyl.

3. A process according to claim 2, wherein R is methyl.

4. (canceled)

5. A process according to claim 1, wherein said alkyl hydrocarbon is n-heptane.

6. A process according to claim 1, wherein the mixture of the two isomers II and V is added dropwise into said alkyl hydrocarbon and said process further comprises stirring the resulting mixture of two isomers and alkyl hydrocarbon.

7. A process according to claim 6, wherein the mixture of two isomers and alkyl hydrocarbon is left stirring for a period of 1-12 hours.

8. A process according to claim 6, wherein the mixture of two isomers and alkyl hydrocarbon is left stirring at a temperature in the range of −80 to 10° C.

9. A process according to claim 1, wherein said alkyl hydrocarbon is present in quantities of 2-50 volumes in relation to the mixture of isomers II and V.

10. A process of preparing fexofenadine according to claim 1, wherein said process farther comprises condensing the compound of formula II, following its separation from the isomer of formula V, in a reaction mixture with azacyclanol.

11. A process according to claim 10, wherein said condensing is carried out in an aprotic organic solvent.

12. A process according to claim 10, wherein said condensing is carried out at a temperature between 40° C. and the reflux temperature of the reaction mixture.

13. A process according to claim 11 wherein the aprotic organic solvent is a ketone.

14. A process according to claim 13, wherein the ketone is methylisobutylketone.
